(19) United States
(12) Patent Application Publication
(10) Pub. No.: US 2007/0072309 A1
(43) Pub. Date: Mar. 29, 2007

(54) Analytical compositions including nanometer-sized transducers, methods to make thereof, and devices therefrom

(75) Inventors: Andrew David Pris, Clifton Park, NY (US); Patrick Ronald Engel, Watertown, MA (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/237,236

(22) Filed: Sep. 29, 2005

(51) Int. Cl. G01N 33/551

(52) U.S. Cl. 436/524

(57) ABSTRACT

Disclosed is an analytical composition comprising a carrier coupled to an analytical transducer nanoparticle and a selector. Disclosed also are methods to make the analytical compositions. The analytical compositions are useful in detection and/or quantification of analytes, such as chemical compounds, biological species, and the like. The selector renders the analytical composition specific to the analyte in question. The detection and/or quantification of the analytical compositions, thus the analytes, occur by virtue of the analytical transducer nanoparticle.

### BACKGROUND

The invention relates generally to analytical compositions, specifically, compositions comprising a carrier coupled linked to a selector and an analytical transducer nanoparticle that are useful to detect analytes. The invention also relates to methods to make the analytical compositions and devices that use the analytical compositions.

The advent of nanometer size materials has allowed for the discovery of new analytical transducers whose unique and desirable characteristics are size dependent. Analytical transducers are materials that are actuated by a stimulus and convert a characteristic of the stimulus to a detectable signal. Analytical transducer nanoparticles are useful in detecting, identifying, and/or quantifying analytes. The steps of detecting, identifying, quantifying, or combinations of the aforementioned steps, are commonly referred to as analytical situations. The transducer nanoparticle is preferably in a stable dispersion in a compatible medium (e.g., water, toluene, or other appropriate solvent). The analytical transducer nanoparticles may be coupled to a selector (e.g., antibodies, chelators, nucleic acids, polymers, or ligands) that possesses specificity to a desired analyte.

This coupling of the analytical transducer nanoparticle to the selector has for the most part, two major inhibitions: (i) relatively small surface area for attachment; and, (ii) aggregation in solution. The first inhibition results in the analytical transducer nanoparticle having a limited number of selector moieties upon its surface and thus reduces the chance of an interaction between the transducer complex and the target to occur. Embodiments of the described invention circumvent this issue by employing carrier particles with a larger surface area, larger than the surface area of the transducer nanoparticle, which the selector can couple to. The resultant construct has a higher selector to analytical transducer nanoparticle ratio, increasing the chances of an interaction between a target and the analytical transducer complex thereby increasing the efficiency of the system. Additionally, the large surface area of the carrier also allows for increased analytical transducer nanoparticle binding, thus increasing signal per recognition event and decreasing noise to signal ratio of the analytical situation.

Embodiments of the invention also provide a way to couple analytical transducer nanoparticles to selectors without resulting in material aggregation of the analytical transducer nanoparticle. Material aggregation of the analytical transducer nanoparticle limits the efficiency of the analytical transducer nanoparticle and increases the variability of the signal/recognition event in the analytical situation. This overall results in the analytical transducer nanoparticle becoming non-optimized and leads to difficulties in quantitating the number of selector events that have occurred. These issues are resolved by the present invention as the non-aggregated transducer-selector-carrier of the present invention reduces background. Also, the inventive non-aggregated transducer-selector-carrier may be used in solution phase assays whereas aggregated constructs may not. The inventive non-aggregated transducer-selector-carrier also may be used in quantitative assays that are non-amenable to specific quantification by aggregated constructs.

### SUMMARY

In one aspect, an analytical composition is provided. The analytical composition comprises a carrier that is coupled to an analytical transducer nanoparticle and a selector.

In another aspect, a method of making an analytical composition is provided. The method comprises providing a carrier, a selector, and an analytical transducer nanoparticle. Subsequently, the carrier and the analytical transducer nanoparticle are reacted to form a coupling between the two moieties. Then, the carrier that is linked to the analytical transducer nanoparticle is reacted with the selector to form a couple between the two moieties. In an alternate embodiment, the carrier and selector are reacted to form a link between the two moieties. Then the carrier that is linked to the selector is reacted with the analytical transducer nanoparticle to form a coupling between the two moieties. In yet another alternate embodiment, the carrier, selector and the analytical transducer nanoparticle are reacted at the same time to form couples between the carrier and selector, and the carrier and the analytical transducer nanoparticle.

In yet another aspect, a device comprising an analytical composition is provided. The device may be used to detect, analyze and quantify analytes. The analytical composition in the device comprises a carrier that is linked to an analytical transducer nanoparticle and a selector.

These and other features, aspects, and advantages of the invention will become better understood when the following detailed description is read with reference to the accompanying figures.

### FIGURES

FIG. 1 schematically illustrates an aspect of the invention wherein a carrier is linked to an analytical transducer nanoparticle and a selector.

FIG. 2 is a flow chart representing exemplary steps for the preparation of an analytical composition in accordance with one aspect of the invention.

FIG. 3 is a flow chart representing exemplary steps for the preparation of an analytical composition in accordance with another aspect of the invention.

FIG. 4 is a flow chart representing exemplary steps for the preparation of an analytical composition in accordance with yet another aspect of the invention.

FIG. 5 illustrates a microparticle complex, carrier particles, amine-modified analytical transducer nanoparticles, and a selector, all exposed to a protein target that was fluorescently labeled.

FIG. 6 illustrates a scanning electron microscopy image of a carrier particle conjugated to fifty nanometer sized analytical transducer nanoparticles.

FIG. 7 illustrates field flow fractionation of carrier particle and analytical transducer nanoparticle reaction mixture.

FIG. 8 illustrates immunoassay selector pairs and expected results.

FIG. 9 illustrates a magnetic particle immunoassay with a carrier particle-analytical transducer nanoparticle-selector complex displaying retained SERS activity and specificity of the selector within the complex.

### DETAILED DESCRIPTION

As used in this specification and the appended claims, the singular forms “a”, “an” and “the” include plural referents unless the context clearly dictates otherwise. “Optional” or “optionally” means that the subsequently described event or circumstance may or may not occur and that the description includes instances where the event or circumstance occurs and instances in which it does not.

“Coupled” or “linked” refers to the interaction between two moieties where the interaction results in a disassociation constant that is less than 10⁻³M. Examples of this type of interaction include: 1) covalent bonds, which is a chemical bond between two atoms or radicals formed by the sharing of at least one pair of electrons; 2) electrostatic interactions or ionic forces, which is the attraction between two oppositely charged moieties; 3) hydrogen bonding, which is the intermolecular couple that exists between a hydrogen atom, that is within a polar bond, and an electron pair that is located on an nearby atom; 4) dipole-dipole attraction, which is the couple between two species that contain a dipole moment; 5) dispersion attraction (or van der Waals forces), where an instantaneous, and opposite, dipole moment is created in a pair of moieties creating a couple; 6) hydrophobic interactions, where two moieties are coupled to one another due to solubility likeness; 7) hydrophilic interactions, where two moieties are couples to one another due to solubility likeness.

The term “functionalized” is used herein to indicate that the described object has a chemical functional group, or chemical species, within its composition that is capable of coupling to another moiety. These functional groups maybe present naturally within the composition or in other cases may have to be added to the objects composition. This can occur through coupling or known chemical transformations. Additionally, already present functional groups maybe transformed to more desirable functionalities through known reaction schemes. As a result of this definition, further functionalization of an object is not necessary if the object already presents the needed chemical groups within its composition, yet the moiety may still be referred to as functionalized. It should be assumed that if not stated, the moiety possesses a functional group that is capable of being coupled.

“Monomers” are organic compounds comprising functional groups that are capable of reacting with themselves, or with other monomers to form “polymers”. “Polymers” are compounds having a plurality of repeating units, and are generally characterized by having molecular weight greater than about 10,000 g/mole. “Oligomers” are compounds having a plurality of repeat units having a molecular weight in the range of from about 500 grams per mole to about 10,000 grams per mole.

“Moiety” refers to a part of a larger composition. “Ligand” refers to a compound that needs to be attached onto another particle to provide selectivity. “Analyte” refers to any detectable species or moiety or moieties that is of interest.

The term “nanoparticle” refers to a particle, generally an organic, semiconductive or metallic particle, having a diameter less than about 1000 nanometer and more preferably less than about 100 nm. Exemplary nanoparticles include, but are not limited to, quantum dots, surface-enhanced Raman scattering particles, surface-enhanced resonance Raman scattering particles, fluorescent dendrimers, carbon nanotubes, paramagnetic particles, fluorescent labeled fullerenes, fluorescent labeled dendrimers, and the like. “Transduction properties” refers to the ability of a particle to be actuated by energy in one form and transforms the energy into another form that may be detected by suitable methods.

It will be appreciated by one of ordinary skill in the art that nanoparticles can exist in a variety of shapes, including but not limited to spheroids, rods, disks, pyramids, cubes, and a plurality of other geometric and non-geometric shapes. While these shapes can affect the physical, optical, and electronic characteristics of nanoparticles, the specific shape does not bear on the qualification of a particle as a nanoparticle.

For convenience, the size of nanoparticles can be described in terms of a “diameter”. In the case of spherically shaped nanoparticle, diameter is used as is commonly understood. For non-spherical nanoparticles, the term diameter, unless otherwise defined, refers to a radius of revolution (e.g., a smallest radius of revolution) in which the entire non-spherical nanoparticle would fit.

A nanoparticle may comprise a “core” of one or more first materials and can optionally be surrounded by a “shell” of a second material. A nanoparticle core surrounded by a shell may be referred to as a “core-shell” nanoparticle.

The term “core” refers to the inner portion of the nanoparticle. A core may substantially include a single homogeneous mono-atomic or polyatomic material. A core can be crystalline, polycrystalline, or amorphous. A core may be “defect” free or contain a range of defect densities. In this case, “defect” can refer to any crystal stacking error, vacancy, insertion, or impurity entity (e.g., a dopant) placed within the material forming the core. Impurities can be atomic or molecular.

While a core may herein be sometimes referred to as “crystalline”, it will be understood by one of ordinary skill in the art that the surface of the core may be polycrystalline or amorphous and that this non-crystalline surface may extend a measurable depth within the core. The potentially non-crystalline nature of the core-surface region does not change what is described herein as a substantially crystalline core. The core-surface region optionally contains defects. The core-surface region will preferably range in depth between one and five atomic-layers and may be substantially homogeneous, substantially inhomogeneous, or continuously varying as a function of position within the core-surface region.

Nanoparticles may optionally comprise a “shell” of a second material that surrounds the core. A shell can include a layer of material, either organic or inorganic, that covers the surface of the core of a nanoparticles. A shell may be crystalline, polycrystalline, or amorphous and optionally comprises dopants or defects.

Shells may be complete, indicating that the shell substantially completely surrounds the outer surface of the core (e.g., substantially all surface atoms of the core are covered with shell material). Alternatively, the shell may be “incomplete” such that the shell partially surrounds the outer surface of the core. In addition, it is possible to create shells of a variety of thicknesses, which can be defined in terms of the number of “monolayers” of shell material that are bound to each core. A “monolayer” is a term known in the art referring to a single complete coating of a shell material (with no additional material added beyond complete coverage). For certain applications, shells will preferably be of a thickness between approximately 0 and 100 monolayers, where it is understood that this range includes non-integer numbers of monolayers. Non-integer numbers of monolayers can correspond to the state in which incomplete monolayers exist. Incomplete monolayers may be either homogeneous or inhomogeneous, forming islands or clumps of shell material on the surface of the nanoparticle. Shells may be either uniform or nonuniform in thickness. In the case of a shell having nonuniform thickness, it is possible to have an “incomplete shell” that contains more than one monolayer of shell material.

It will be understood by one of ordinary skill in the art that there is typically a region between the core and shell referred to as an “interface region”. The interface region may comprise an atomically discrete transition between the material of the core and the material of the shell or may comprise an alloy of the materials of the core and shell. The interface region may be lattice-matched or unmatched and may be crystalline or noncrystalline. The interface region may contain one or more defects or be defect-free. The interface region may be homogeneous or heterogeneous and may comprise chemical characteristics that are graded between the core and shell materials such that a gradual or continuous transition is made between the core and the shell. Alternatively, the transition can be discontinuous. The width of the interface region can range from an atomically discrete transition to a continuous graded alloy of core and shell materials that are purely core material in the center of the quantum dot and purely shell material at the outer surface.

The term “shell” is used herein to describe shells formed from substantially one material as well as a plurality of materials that can, for example, be arranged as multi-layer shells. A shell may optionally comprise multiple layers of a plurality of materials in an onion-like structure, such that each material acts as a shell for the next-most inner layer. Between each layer there is, optionally, an interface region.

The term “peptide” refers to oligomers or polymers of any length wherein the constituent monomers are alpha amino acids linked through amide bonds, and encompasses amino acid dimers as well as polypeptides, peptide fragments, peptide analogs, naturally occurring proteins, mutated, variant or chemically modified proteins, fusion proteins, and the like. The amino acids of the peptide molecules may be any of the twenty conventional amino acids, stereoisomers (e.g., D-amino acids) of the conventional amino acids, structural variants of the conventional amino acids, e.g., iso-valine, or non-naturally occurring amino acids such as α,α-disubstituted amino acids, N-alkyl amino acids, β-alanine, naphthylalanine, 3-pyridylalanine, 4-hydroxyproline, O-phosphoserine, N-acetylserine, N-formylmethionine, 3-methylhistidine, 5-hydroxylysine, and nor-leucine. In addition, the term “peptide” encompasses peptides with posttranslational modifications such as glycosylations, acetylations, phosphorylations, and the like.

The term “oligonucleotide” is used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the oligonucleotide. More particularly, the term includes polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholine (commercially available from the Anti-Virals, Inc., Corvallis, Oreg., as Neugene) polymers, and other synthetic sequence-specific nucleic acid polymers, providing that the polymers contain nucleobases in a configuration that allows for base pairing and base stacking, such as is found in DNA and RNA. There is no intended distinction in length between the terms “polynucleotide”, “oligonucleotide”, “nucleic acid” and “nucleic acid molecule”, and these terms refer only to the primary structure of the molecule. Thus, these terms include, for example, 3′-deoxy-2′,5′-DNA, oligodeoxyribonucleotide N3′P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps”, substitution of one or more of the naturally occurring nucleotides with an analog, internucleotide modifications such as, for, example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes other kinds of nucleic acids such as, but not limited to, locked nucleic acids (LNAs).

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

Furthermore, modifications to nucleotidic units include rearranging, appending, substituting for or otherwise altering functional groups on the purine or pyrimidine base that form hydrogen bonds to a respective complementary pyrimidine or purine. The resultant modified nucleotidic unit optionally may form a base pair with other such modified nucleotidic units but not with A, T, C, G or U. Basic sites may be incorporated which do not prevent the function of the polynucleotide. Some or all of the residues in the polynucleotide can optionally be modified in one or more ways.

The term “antibody” as used herein includes antibodies obtained from both polyclonal and monoclonal preparations, as well as: hybrid (chimeric) antibody molecules (see, for example, Winter et al. (1991), Nature, volume 349, pp. 293-299; and U.S. Pat. No. 4,816,567); F(ab′)2 and F(ab) fragments; Fv molecules (noncovalent heterodimers); single-chain Fv molecules (sFv); dimeric and trimeric antibody fragment constructs; minibodies; humanized antibody molecules; and, any functional fragments obtained from such molecules, wherein such fragments retain specific-binding properties of the parent antibody molecule.

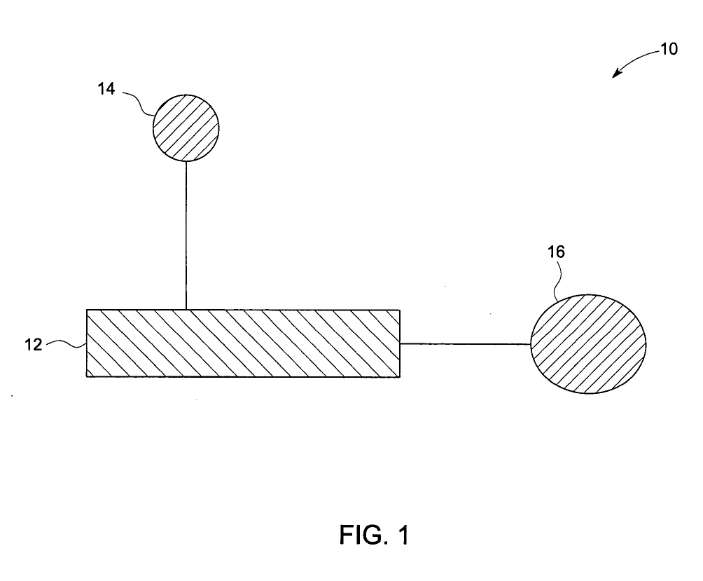

FIG. 1 illustrates an analytical composition designated by the reference numeral 10, which includes a functionalized carrier 12 that is linked to both a functionalized analytical transducer nanoparticle 14 and a selector 16. The links between the functionalized carrier and the functionalized analytical transducer nanoparticle may be alkane, alkene, alkyne, amide, amine, hydazone, Schiff base, amidine, arylamine, carbamate, isourea, ester, di-sulfide, isothiourea, thioester, ether, epoxide, epoxy, thioether, sulfonamide, phosphoramidate, and combinations of the foregoing bonds. Similarly, the coupling between the functionalized carrier and the functionalized selector may be alkane, alkene, alkyne, amide, amine, hydazone, Schiff base, amidine, arylamine, carbamate, isourea, ester, di-sulfide, isothiourea, thioester, ether, epoxide, epoxy, thioether, sulfonamide, phosphoramidate, and combinations of the foregoing bonds.

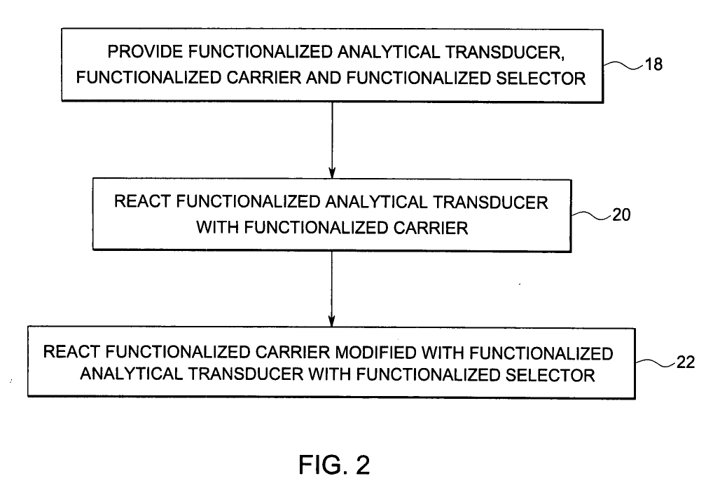

Referring to FIG. 2, a method for making an analytical composition is illustrated. Initially, a functionalized analytical transducer nanoparticle, a functionalized carrier and a selector are provided, as represented by block 18. Subsequently, the functionalized analytical transducer nanoparticle is reacted with the functionalized carrier to form the links between them, as represented by block 20. Next, the functionalized carrier that has been modified with functionalized analytical transducer nanoparticle is reacted with selector to form links between the functionalized carrier and the selector, as represented by block 22.

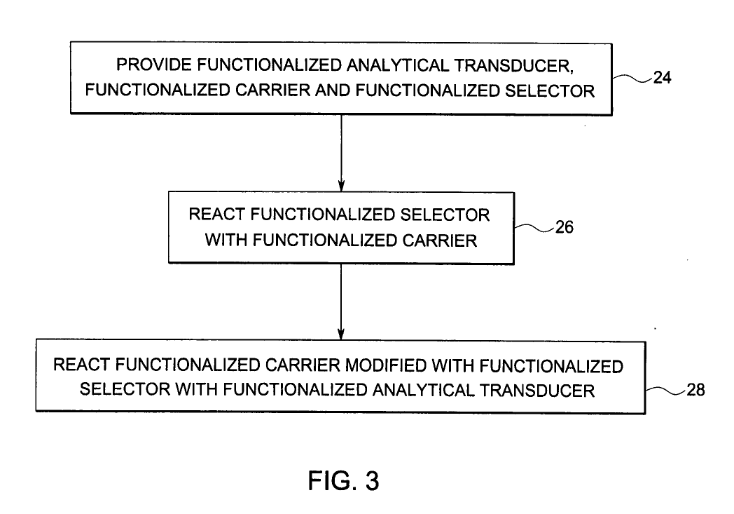

FIG. 3 illustrates another method for making an analytical composition. Initially, a functionalized analytical transducer nanoparticle, a functionalized carrier and a selector are provided, as represented by block 24. Subsequently, the selector is reacted with the functionalized carrier to form the links between them, as represented by block 26. Next, the functionalized carrier that has been modified with selector is reacted with functionalized analytical transducer nanoparticle to form links between the functionalized carrier and the functionalized analytical transducer nanoparticle, as represented by block 28.

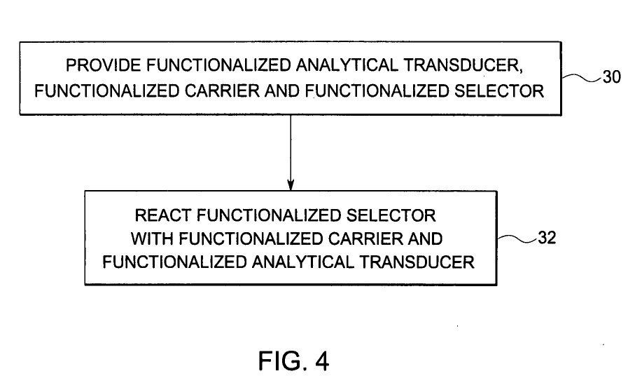

FIG. 4 illustrates yet another method for making an analytical composition. Initially, a functionalized analytical transducer nanoparticle, a functionalized carrier, and a selector are provided as represented by block 30. Subsequently, the functionalized carrier, the selector, and the functionalized analytical transducer nanoparticle are reacted with each other simultaneously, to form the links between them, as represented by block 32.

Functionalized carriers may be selected from a wide range of carrier materials that are functionalized or are not functionalized. The carrier materials preferably are at least as large as 100 nm and may be as large as 1000 nm. These carrier materials can include, for example, organic and inorganic polymers or glasses with different properties including mechanical strength, optical transparency, light wave transmissivity, thermal stability, dimensional stability, low temperature flexibility, moisture absorption, and chemical inertness. Some exemplary materials that can be made in the micron particle size range include, but not limited to, alumina, iron oxide, titanium oxide, silica, glass, tin oxide, and the like. Polymeric materials may also be used for this purpose. Some exemplary carriers may be selected from a group of polymers consisting of fluoropolymers (e.g., Teflon AF) brand fluoropolymers available from DuPont, Cytop® brand fluoropolymers available from Asahi), polymers derived from B-staged bisbenzocyclobutene monomers (e.g., Cyclotene® brand resins and Cyclotene® brand fluorinated resins available from Dow Chemical Company), phenolic resin, and fluorinated poly(aryl ether sulfide), poly(isobutylene), poly(diphenoxyphosphazene), fluorinated acrylate (ZPU series from Zen Photonics Co., LTD), poly(methyl methacrylate), poly(vinyl alcohol), poly(vinyl butyral), poly(vinylcarbazole), poly(vinyl fluoride), poly(methyl vinyl ether), polyethylene, polypropylene, polystyrene, poly(vinyl pyridine), poly(ethylene oxide), fluorinated acrylates, poly(siloxanes), poly(silanes), poly(diphenoxyphosphazenes), poly(vinyl ferrocene), polycarbonates, poly(cyclic olefin) such as Zenor® and Zenex® and the like. Copolymers, including random and block copolymers, cross-linkable polymers, and blends of two or more polymers are also contemplated for use as carrier materials.

Carrier materials may comprise functional groups that are accessible for reaction with other functional groups to form linkages. Functional groups may include any of the organic functional groups that are known to those skilled in the art. Suitable functional groups include, but are not limited to, acetals, ketals, acetylenic linkages, halides (e.g., acid chlorides, sulfonyl halides, alkyl halides, haloacetyl, arylhaloside), alcohols, aldehydes, ethylenic linkages (e.g., vinyl, acryloyl derivitives), esters, amides, amines, carboxylic acids, carboxylic anhydrides, azo groups (e.g., diazoalkane, diazoacetyl), boranes, carbamates, epoxides, glycidyl ethers, glycidyl esters, thioethers, thiols, di-sulfides, cyano linkages, isothiocyanates, isocyanates, nitro groups, sulfonyl halides, sulfoxides, phenols, thiophenols, aromatics, hydrazides, aryl azides, nitrenes, imidoesters, benzophenones, carbonyldiimidazoles, carbodiimides, aziridines, alkylphosphates, siloxanes, and the like. The carrier materials may be suitably functionalized to facilitate reaction with functionalized nanoparticles. Therefore, high contents of functionalized nanoparticles can be readily incorporated onto the carrier material while maintaining desired uniformity and homogeneity. More importantly, the properties of the final composition can be tuned for a variety of applications primarily by adjusting the content of the functionalized nanoparticles and by selecting the carrier material. The chosen carrier material desirably should meet other requirements for a specific application. Functionalized carriers may be prepared by conventional methods known to those skilled in the art, or may be commercially available from a variety of sources.

Nanoparticles may be fabricated using a variety of nucleation and growth wet chemistries (such as but not limited to Turkevich syntheisis, Stober synthesis, or emulsion polymerization) as well as vapor deposition, ion-implantation, photolithography, spatially modulated electric fields, semiconductor doped glasses, strain-induced potential variations in quantum wells, atomic width fluctuations in quantum wells, and a variety of other techniques. The nanoparticles of the invention are particles having a diameter less than about 1000 nm, preferably less than about 100 nm, more preferably less than about 50 nm.

Semiconductive nanoparticles may be composed of an organic semiconductor material or an inorganic semiconductor material. Organic semiconductor materials may be conjugated polymers. Suitable conjugated polymers include, for example, cis and trans polyacetylenes, polydiacetylenes, polyparaphenylenes, polypyrroles, polythiophenes, polybithiophenes, polyisothianaphthene, polythienylvinylenes, polyphenylenesulfide, polyaniline, polyphenylenevinylenes, and polyphenylenevinylene derivatives, e.g., poly(2-methoxy-5-(2-ethylhexyloxy)-1,4-phenylene vinylene (“NMH-PPV”) (see U.S. Pat. No. 5,189,136 to Wudl et al.), poly(2,5-bis-chelostanoxy-1,4-phenylene vinylene) (“BCHA-PPV”) (e.g., as described in International Patent Publication No. WO 98/27136), and poly(2-N,N-dimethylamino phenylene vinylene)(described in U.S. Pat. No. 5,604,292 to Stenger-Smith et al.). In particular, exemplary materials for use as semiconductor nanocrystals in the biological and chemical assays of the present invention include, but are not limited to, those described above, including semiconductors such as ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AlS, AlP, AlSb, PbS, PbSe, Ge, Si and ternary and quaternary mixtures thereof. For any particular nanocrystal composition, it is also possible to tune the emission to a desired wavelength by controlling the particle size distribution.

The analytical transducer nanoparticles of the invention may also be metallic nanoparticles. Such particles are useful, for example, in surface enhanced Raman scattering (SERS), which employs nanometer-sized particles onto which Raman active moieties (e.g., a dye or pigment, or a functional group exhibiting a characteristic Raman spectrum) are adsorbed or attached. Metallic nanoparticles may be comprised of any metal or metallic alloy or composite, although for use in SERS, a SERS active metal is used, e.g., silver, gold, copper, lithium, aluminum, platinum, palladium, or the like. In addition, the particles can be in a core-shell configuration, e.g., a gold core may be encased in a silver shell, or the particles may form small aggregates in solution.

SERS particles may also be organic molecules that have distinctive Raman scattering patterns. Examples of such molecules include, but are not limited to, dithiobisbenzoic acid, 4-mercaptobenzoic acid, 2-naphthalenethiol, thiophenol, direct red 81, Chicago sky blue, 4,4′-dithiobis(succinimidylbenzoate), p-dimethylaminoazobenzene, 1,5-difluoro-2,4-dinitrobenzene, 4-(4-aminophenylazo)phenylarsonic acid monosodium salt, arsenazo I, basic fuchsin, disperse orange 3, 2-(4-hydroxyphenylazo)-benzoic acid, erythrosine B, tryptan blue, ponceau S, ponceau SS, 5,5′-dithiobis(2-nitrobenzoic acid), polymeric particles, and the like. Variations of SERS particles, such as surface enhanced resonance Raman spectroscopy (SERRS), are contemplated in some aspects of the invention.

Analytical transducer nanoparticles of the invention may also be nanoparticles that incorporate fluorophores. Typical fluorophores that may be used include, but are not limited to, fluorescein, rhodamine, dansyl, fluorescamine, pyrene, acridine, 2-methoxy-2,4-diphenyl-3(2H)-furanone, and the like.

In a further embodiment, analytical transducer nanoparticles may include superparamagnetic nanoparticles. Superparamagnetic nanoparticles are magnetic materials that do not substantially show magnetization at zero applied magnetic fields. Upon an application of a magnetic field, these nanoparticles align themselves along the axis of the magnetic field. Commonly used superparamagnetic nanoparticles include, but are not limited to, yttrium ion garnet, iron oxide, and the like.

Further embodiments of the invention include the use of fluorescent fullerenes, fluorescent dendrimers, carbon nanotubes, and the like to be used as analytical transducers.

The surface of the analytical transducer nanoparticle may be modified with an over coating layer or shell layer. A shell or coating for the analytical transducer is desirable, as surface defects can trap electrons that degrade the electrical and optical properties of the nanoparticle. An insulating layer at the surface of the analytical transducer nanoparticle provides an atomically abrupt jump in the chemical potential at the interface that eliminates energy states that can serve as traps for electrons, thus increasing efficiency in the signal generation process. The shell may also be used for imparting some other physical and chemical properties to the analytical transducer nanoparticles, such as functionalization.

The functional groups may be incorporated onto the surface of the analytical transducer nanoparticles, typically, as part of the shell layer of the analytical transducer nanoparticle. Preferably, functionalized analytical transducer nanoparticles are formed or used in a form that can be easily incorporated into devices. According to some embodiments of the invention, a unique physical characteristic of functionalized analytical transducer nanoparticles is that, while the core can comprise a crystalline semiconductor material, the surface can be coated with a variety of different organic and/or inorganic materials. These surface coatings (e.g., shells or ligand layers) can impart stability and/or chemical activity, as well as passivation of electrically and optically active defect sites on the surface. These surface coatings are optionally substantially different in chemical nature than the inorganic core. As a result, while functionalized analytical transducer nanoparticles can comprise primarily a highly nonlinear semiconductor material, they display the surface characteristics to the surrounding material.

In one embodiment, the surface functionalizations are preferably bi-functional. The term bi-functional is meant to convey that there are at least two portions of the surface functionality such that one portion interacts primarily with the functionalized analytical transducer nanoparticles surface, while the second portion interacts primarily with the surrounding environment (e.g., solvent, carrier material etc.). These at least two portions of the surface functionality may be the same or different, contiguous or noncontiguous, and are optionally contained within two or more different molecular species that interact with each other. The at least two portions may include hydrophilic groups, hydrophobic groups, or amphiphilic groups. The interaction of each of the at least two portions and the functionalized analytical transducer nanoparticles or surrounding environment can be covalent or noncovalent, strongly interacting or weakly interacting, and can be labile or non-labile. The at least two portions can be selected independently or together.

In some embodiments, the surface functionalities are employed such that the portion that interacts with the functionalized analytical transducer nanoparticles passivates defects on the surface such that the surface is made substantially defect-free. Also, the portion that interacts with the environment may be selected to impart stability and compatibility (e.g., chemical compatibility or affinity) for a specific application. Alternative methods of achieving these requirements include (but are not limited to): 1) passivating the surface of the functionalized analytical transducer nanoparticles independent of the surface layer (e.g., using a shell or creating an intrinsically defect free surface), while the environmental compatibility is imparted by the surface functionalities, or 2) imparting both passivation and environmental compatibility independent of the surface layer.

Through the appropriate selection of surface functionalities, functionalized analytical transducer nanoparticles can be reacted with a variety of carrier materials such as, for example, glasses, polymers, crystalline solids, and the like.

One aspect of the invention relates to effectively maintaining the electronic properties of the functionalized analytical transducer nanoparticles independent from the optical, chemical, mechanical, and other properties of the functionalized carrier material coupled with the functionalized nanoparticles. In such an aspect, it is possible to combine the large electronic properties of functionalized analytical transducer nanoparticles with the ease of handling and processability of a carrier material.

In addition to conveying stability and chemical compatibility with the surrounding environment, the surface functionalization can optionally be used to tailor the physical, optical, chemical, and other properties of the analytical transducer nanoparticles themselves. In some cases, the surface functionality can be electrically active, optically active, physically active, chemically active, or a combination thereof. Active species are used to precisely control the electrical, optical, transport, chemical, and physical interactions between analytical transducer nanoparticles and the surrounding environment and/or the properties of individual analytical transducer nanoparticles. For instance, a conjugated bond covalently bound to the surface of one or more analytical transducer nanoparticles may facilitate charge transfer out of one analytical transducer nanoparticle and into another. Similarly, a physically rigid active group bound in a geometry substantially normal to the surface of a analytical transducer nanoparticle can act as a physical spacer, precisely controlling minimum interparticle spacing within an engineered material.

In some embodiments, interactions between individual analytical transducer nanoparticles using surface functionalities that foster interactions between analytical transducer nanoparticles are increased. Surface functionalities contact surface functionalities from other analytical transducer nanoparticles or with other analytical transducer nanoparticles directly, and increase the degree of collective phenomena compared to single particle phenomena.

Surface functionalities comprising chemically active functional groups allow analytical transducer nanoparticles to be attached to carrier materials, along with other optional active molecules. This provides a method for controlling the density of analytical transducer nanoparticles within close proximity of molecules that influence a variety of functions such as carrier transport or delocalization. Suitable functional groups include, but are not limited to, acetals, ketals, acetylenic linkages, halides (e.g., acid chlorides, sulfonyl halides, alkyl halides, haloacetyl, arylhaloside), alcohols, aldehydes, ethylenic linkages (e.g., vinyl, acryloyl derivitives), esters, amides, amines, carboxylic acids, carboxylic anhydrides, azo groups (e.g., diazoalkane, diazoacetyl), boranes, carbamates, epoxides, glycidyl ethers, glycidyl esters, thioethers, thiols, di-sulfides, cyano linkages, isothiocyanates, isocyanates, nitro groups, sulfonyl halides, sulfoxides, phenols, thiophenols, aromatics, hydrazides, aryl azides, nitrenes, imidoesters, benzophenones, carbonyldiimidazoles, carbodiimides, aziridines, alkylphosphates, siloxanes, and the like. The analytical transducer nanoparticles may be suitably functionalized to facilitate reaction with functionalized carrier materials.

Surface functionalities may comprise species such as, but not limited to, conducting polymers, charge transfer species, conjugated polymers, aromatic compounds, or molecules with donor-acceptor pairs. These surface functionalities can foster electron delocalization or transport and thus can increase the interaction between analytical transducer nanoparticles. Additionally, the surface functionalities may be selected to facilitate high quantum dot number densities without the detrimental aggregation that often plagues high concentration systems. Surface functionalities can also be selected to impart stability of analytical transducer nanoparticles under a variety of environmental conditions including ambient conditions.

Those embodiments where the analytical transducer nanoparticles are not desirably functionalized, they may be functionalized by reaction with a reagent that contains a functional group capable of reacting with the shell layer and another functional group that provides the functional characteristic to the analytical transducer nanoparticle. In some embodiments, the two functional groups are separate. In alternative embodiments, the two functional groups are part of a single moiety.

Coupling between the carrier and the analytical transducer nanoparticle may be carried out using standard reaction sequences known to those skilled in the art. The electronic properties of the analytical transducer nanoparticles are retained and/or tuned by the functionalization step as well as the reaction with functionalized carrier step. The functional groups that result from reacting the functionalized analytical transducer nanoparticles and the functionalized carrier material may include, but are not limited to, alkane, alkene, alkyne, amide, amine, hydazone, Schiff base, amidine, arylamine, carbamate, isourea, ester, disulfide, isothiourea, thioester, ether, epoxide, epoxy, thioether, sulfonamide, phosphoramidate, and combinations of the foregoing bonds.

In some embodiments, more than one analytical transducer nanoparticle that is linked to a carrier is present per carrier, improving the detection signal from the analytical composition. In some embodiments, the selector material, the functionalized carrier and functionalized analytical transducer nanoparticle combine to form an analytical composition that interact with an analyte, thereby detecting compounds, biological processes or reactions, and/or altering biological molecules or processes. Preferably, the interaction of the functionalized selector material and the analyte involves specific binding.

The selector material associated with the carrier and the analytical transducer nanoparticle can be naturally occurring or chemically synthesized. The selector employed may have desired physical, chemical, or biological properties, including, but not limited to, covalent and noncovalent association with proteins, nucleic acids, signaling molecules, prokaryotic or eukaryotic cells, viruses, subcellular organelles and any other biological and chemical compounds. Selectors may also be the ability to affect a biological process (e.g. cell cycle, blood coagulation, cell death, transcription, translation, signal transduction, DNA damage or cleavage, production of radicals, scavenging radicals, etc.), or alter the structure of a biological compound (e.g. crosslinking, proteolytic cleavage, radical damage, etc.).

Selector materials typically possess a functional group inherently, as part of the structure. Alternately, the functional group may be introduced by chemical modification of the selector material. Methods to introduce chemical functional groups onto the selector material are know and widely used in the art. Functional groups may comprise any one of acetals, ketals, acetylenic linkages, halides (e.g., acid chlorides, sulfonyl halides, alkyl halides, haloacetyl, arylhaloside), alcohols, aldehydes, ethylenic linkages (e.g., vinyl, acryloyl derivitives), esters, amides, amines, carboxylic acids, carboxylic anhydrides, azo groups (e.g., diazoalkane, diazoacetyl), boranes, carbamates, epoxides, glycidyl ethers, glycidyl esters, thioethers, thiols, di-sulfides, cyano linkages, isothiocyanates, isocyanates, nitro groups, sulfonyl halides, sulfoxides, phenols, thiophenols, aromatics, hydrazide, aryl azides, nitrenes, imidoesters, benzophenones, carbonyldimidazoles, carbodiimides, aziridines, alkylphosphates, siloxanes, and the like, and combinations thereof.

The analytical composition of embodiments of the invention comprises a functionalized analytical transducer nanoparticle linked to a functionalized carrier, and the functionalized carrier linked to a selector material. The selector material may be a nucleic acid such as any ribonucleic acid, deoxyribonucleic acid, dideoxyribonucleic acid, or any derivatives and combinations thereof. The nucleic acid can also be oligonucleotides of any length. The oligonucleotides can be single-stranded, double-stranded, triple-stranded or higher order configurations.

Among the preferred uses of the present compositions and methods are detecting and/or quantitating nucleic acids as follows: (a) viral nucleic acids; (b) bacterial nucleic acids; and (c) numerous human sequences of interest, e.g. single nucleotide polymorphisms. Analytical compositions may comprise individual nucleotides, deoxynucleotides, dideoxynucleotides or any derivatives and combinations thereof. Nucleotides also include monophosphate, diphosphate and triphosphates and cyclic derivatives such as cyclic adenine monophosphate (cAMP). The selector material may also include enzymes, enzyme substrates, enzyme inhibitors, cellular organelles, lipids, phospholipids, fatty acids, sterols, cell membranes, molecules involved in signal transduction, receptors and ion channels.

Standard reaction sequences required to link the selector are known to those skilled in the art and may be followed to achieve the same. Typical functional groups that arise from reacting the selector material and the carrier material include, but are not limited to, alkane, alkene, alkyne, amide, amine, hydazone, Schiff base, amidine, arylamine, carbamate, isourea, ester, disulfide, isothiourea, thioester, ether, epoxide, epoxy, thioether, sulfonamide, phosphoramidate, and combinations of the foregoing bonds

The spatial order of attachment of the analytical transducer nanoparticle and the selector onto the carrier can be varied. Thus, in one embodiment, the selector is linked to the carrier, following which the analytical transducer nanoparticle is linked to the carrier that is already coupled to the selector. In an alternate embodiment, the analytical transducer nanoparticle is linked to the carrier, following which the selector is linked to the carrier that is already coupled to the analytical transducer nanoparticle. Likewise, the temporal order of attachment may also be varied. Thus, the three elements can be coupled simultaneously or in sequence with any two elements being coupled followed by the addition of the third element.

The analytical composition of the invention is useful for detection of a specific analyte by signal transduction. Upon exposure of the analytical composition to energy, some or all of the energy may be transferred to the analytical transducer nanoparticle, which generates one or more detectable signal(s). These detectable signals may include (1) emission of electromagnetic radiation, (2) absorption of radiation, and (3) scattering, including diffraction, of radiation.

The emission properties of the analytical composition may be useful in a variety of applications. The analytical transducer nanoparticles, may be capable of being excited over a broad bandwidth, while exhibiting emission in a narrow wavelength band; thus electromagnetic radiation of wavelength ranging from x-ray to ultraviolet to visible to infrared waves may be used to excite the nanoparticles. In addition, the analytical transducer nanoparticles are capable of excitation from bombardment with a particle beam such as an electron beam (e-beam). Furthermore, because of the broad bandwidth at which the analytical transducer nanoparticles are excitable, one may use a common excitation source for the simultaneous excitation of several analytical transducers, e.g., several analytical transducers which give off radiation at different frequencies, thus permitting simultaneous excitation and detection of the presence of several analytical transducers indicating, for example, the presence of several analytes in the material being examined.

Another detectable signal provided by the analytical transducer nanoparticle in response to radiation is absorption. The presence of the analytical composition may be indicated by detection of absorption of radiation. Since the analytical transducer nanoparticle has such a wide wavelength band of absorption, detection of the analytical composition may be carried out over a wide range of wavelengths, according to the requirements of the detection process. The analytical composition of the invention may also provide a detectable scattering signal in response to exposure to energy. This detectable scattering signal may be a diffraction signal.

A further use of the analytical composition of the invention is to provide a detectable signal in response to energy transferred from one or more spatially proximal sources. In this context, “energy transfer” is meant the transfer of energy from one atom, molecule, or any other substance (e.g. a polymer, a gel, a lipid bilayer, etc.) to another atom, molecule, or any other substance by either (1) a radiative pathway (e.g., emission of radiation by a first atom or molecule followed by scattering—including diffraction—and/or absorption of the emitted radiation by a second atom or molecule); or (2) a non-radiative pathway (e.g., fluorescence resonance energy transfer, or FRET, from a first atom or molecule to a second atom or molecule). By use of the term “proximal source” is meant an atom, a molecule, or any other substance which is capable of transferring energy to and/or receiving energy transferred from another atom or molecule or any other substance. The term “spatially proximal source” refers to a proximal source spaced sufficiently close to enable energy to be transferred from a proximal source to an analytical composition. For example, in the case of FRET, a spatially proximal source comprises a proximal source spaced 10 nm or less from the analytical composition. In the case of the transfer of radioactive energy, a spatially proximal source comprises a proximal source spaced 1 micron or less from the analytical composition. The intensity of the detected signal indicates the presence of analytes, proximity of the analytes to the analytical composition, concentration of the analytes, and the like.

The selectivity of the analytical compositions of the invention is largely due to the selector materials used. The selector materials are chosen based on the specificity of the selector to a certain kind of chemical compound, cell, antibody, protein, amino acids, and the like that is desired to be detected and/or quantified. In many embodiments, the analytical composition can bind itself to analytes by virtue of the presence of the selector material. The quantity of the selector material and the analytical transducer nanoparticle in the analytical composition may be chosen such that even trace amounts of analytes may give rise to an easily detectable signal is made available from the composition.

In general, the analytical composition may be used to detect the presence of analytes by introducing the analytical composition dispersed in the material, permitting the selector material of the analytical composition to bond to the analytes that may be present in the material. After introduction of the analytical composition into the material, uncoupled analytical compositions may be optionally removed from the material, leaving only bonded analytical compositions. Alternately, after introduction of the analytical composition into the material, uncoupled analytical compositions may be left in the system. In either event, the material may be exposed to an energy source capable of causing the analytical compositions to provide a detectable signal.

The presence of the larger sized carrier material increases the overall size of the analytical composition. This serves to improve the stability of the dispersion of analytical compositions in a suitable medium, such as an aqueous medium, as delineated by the Derjaguin-Landau-Verway-Overbeek (DLVO) theory. Thus, the analytical composition described in the invention combine the advantages of (i) greater number of analytical transducer nanoparticles in the analytical composition resulting in ease of detection; (ii) increased selectivity of the composition towards the analytes due to greater number of selector materials; (iii) increased stability of the dispersion in the medium due to greater particle size of the final analytical composition.

### EXAMPLES

In this Example, all steps were carried out using silane treated microcentrifuge tubes. To aid in the understanding of embodiments of the invention, a method through which a specific analytical transducer nanoparticle may be created and chemically modified is described.

### Example 1

### Production of the Transducer-Selector-Carrier Complex

A. Functionalization of the Analytical Transducers: Analytical transducer nanoparticles were created using the method of Mulvaney, et al., Glass-coated, analyte-tagged nanoparticles: A new tagging system based on detection with surface-enhanced Raman scattering. Langmuir 2003, 19, (11), 4784-4790, incorporated herein by reference. This synthesis creates glass-coated, analyte-tagged nanoparticles known as GANs (which may be used as the analytical nanoparticle in embodiments of the invention). GANs possess surface enhanced Raman scattering (SERS) activity that provides distinctive SERS spectra upon specific illumination, which is suited for the particular particle size and the particular Raman tag. For use in this Example the Raman scatter incorporated into the GANs was trans-bis(pyridyl)ethylene which provided a SERS spectrum when illuminated with 785-nm light.

The analytical transducer nanoparticles were prepared for conjugation by reacting the siloxane groups on the nanoparticles in a pH 9.5 buffer with a 10× molar excess of 3-aminopropyltrimethoxysilane for 90 minutes. The resultant functionalized amine modified analytical transducer nanoparticles were washed with and resuspended in de-ionized water by centrifugation (5000 rcf for 5 minutes).

B. Preparation of the Functional Carrier. In this Example, pre-functionalized polystyrene (possessing available carboxylate groups) particles were used as the carrier; however, any agent with a size greater than 200 nm and capable of coupling to the transducer nanoparticle and the selection may be used. 300 μL of carboxylate/sulfate-modified polystyrene particles (PS—COO⁻/SO₃⁻, surfactant free, Interfacial Dynamics Corp.) were added.

C. Combining the Selector. Transducer, and Carrier: The transducer nanoparticle (200 μL from Step A) and functionalized carrier (300 μL from Step B) were introduced into 300 μL of cold 25 mM 2-morpholinoethanesulfonic acid buffer (MES, pH 6.0, Sigma), and 50 μL the 2 mg/mL IgG specific for the target analyte. Mouse anti-goat IgG (Pierce) was the selector use in this Example; however, any agent capable of specifically binding the target analyte may be used and capable of coupling to the carrier. The mixture was rotated for 10 minutes at 4° C., and 120 μL of 50 mg/mL N-hydroxysulfosuccinimide (sulfo-NHS, Pierce) and 120 μL of 50 mg/mL 1-ethyl-3-(3-dimethylaminopropyl)carbodiimide hydrochloride (EDC, Pierce) (both dissolved in cold 25 mM MES, pH 6.0) were added.

The transducer nanoparticle, selector, and carrier coupled following overnight at 4° C. through incubation under gentle agitation. The reactants were separated from the products by transferring the solution to a 1.5 mL microcentrifuge tube and centrifuging at 2000 rcf for 4 minutes. Contents of the tube to formed three layers: (1) the upper layer consisting of the suspended white un-reacted polystyrene nanoparticles; (2) a pink-colored, loosely packed middle layer contained the transducer-selector-carrier complex and (3) a hard, red/purple pellet of un-reacted glass-coated analytical transducer nanoparticles at the bottom of the tube (which remain aggregated when centrifuged in this buffer). The white suspension upper layer was removed and replaced with 600 μL of high ionic strength buffer containing a surfactant, 100 mM sodium phosphate, 150 mM sodium chloride, 1% BSA, 0.01% Tween-20 buffer (pH 7.5). The loose middle layer was resuspended through mild agitation while the hard pellet remained in pellet form following agitation.

The transducer-selector-carrier complex isolated in the first wash may be used. However, purity and performance is enhanced by additional washing steps to remove excess, unbound reactants. The pink re-suspended middle layer was withdrawn and placed into another 1.5 mL microcentrifuge tube. Following agitation, the solution again separated into three layers. The middle layer again centrifuged at 2000 rcf for 4 minutes, after which only a pink loose pellet is formed. In some cases a slight presence of white particles are found in the upper layer. The supernatant with the white suspended particles was removed and replaced with 600 μL of 100 mM sodium phosphate, 150 mM sodium chloride, 1% BSA, 0.01% Tween-20 buffer (pH 7.5). The pink layer is again resuspended using slight agitation and centrifuged for a third time at 2000 rcf for 4 minutes. The now clear-supernatant is removed and replaced with 300 μL of 100 mM sodium phosphate, 150 mM sodium chloride, 1% BSA, 0.01% Tween-20 (Sigma) buffer (pH 7.5). The product, supernatant containing the transducer-selector-carrier complex may be stored at 4° C.

### Example 2

### Immunoassay to Demonstrate Complex Formation and Specific Binding

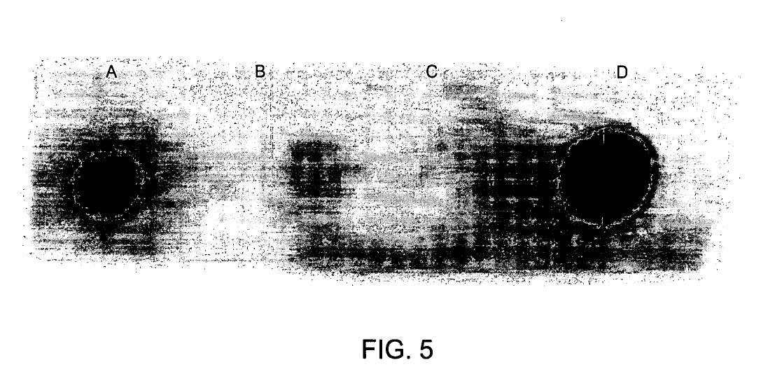

As illustrated in FIG. 5, mouse anti-goat antibody coupled to fluorescent goat anti-mouse antibody (AlexaFlour488™) was used to screen four separate dot-blots. Dot blot A contained the transducer-selector-carrier complex; dot blot B contained the polystyrene carrier alone; dot blot C contained the transducer nanoparticle alone; and dot blot D contained the selector alone (rabbit anti-mouse antibody).

The results shown in FIG. 5 demonstrate that the microparticle complex contains active selectors (see FIG. 5A) upon its surface. The dot blot assay shown in FIGS. 5B and 5C show that the carrier particle and transducer nanoparticle, respectively, do not bind to the labeling antibodies, supporting the conclusion that binding was specific for the selector. The dot blot shown in FIG. 5D is the positive control.

### Example 3

### Scanning Electron Microscope Imaging of the Transducer-Selector-Carrier Complex Demonstrate Coupling

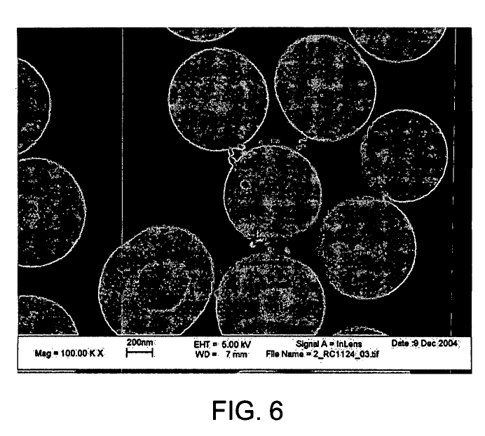

FIG. 6 depicts a scanning electron microscope (SEM) image of the product of Example 1. This image was obtained by first exchanging the transducer-selector-carrier complex to pure water from the high ionic strength buffer through centrifugation of the complex at 2000 rcf for 4 minutes, decanting the supernatant and replacing that volume with pure water. The complex was resuspended through mild agitation. This process was completed three times in total and was completed in order to prevent salt crystal formation on the imaged substrate. At this point, a small volume of the transducer-selector-carrier complex was dried upon a gold substrate and the sample was submitted for standard SEM imaging.

FIG. 6 illustrates that the smaller (˜50-nm) analytical transducer nanoparticles are bound to the larger carrier particles. Moreover, there are multiple analytical transducers bound per carrier particle. This will result in increased signal per recognition event as compared to directly binding the selector to the analytical transducer nanoparticle.

### Example 4

### Field Flow Fractionation

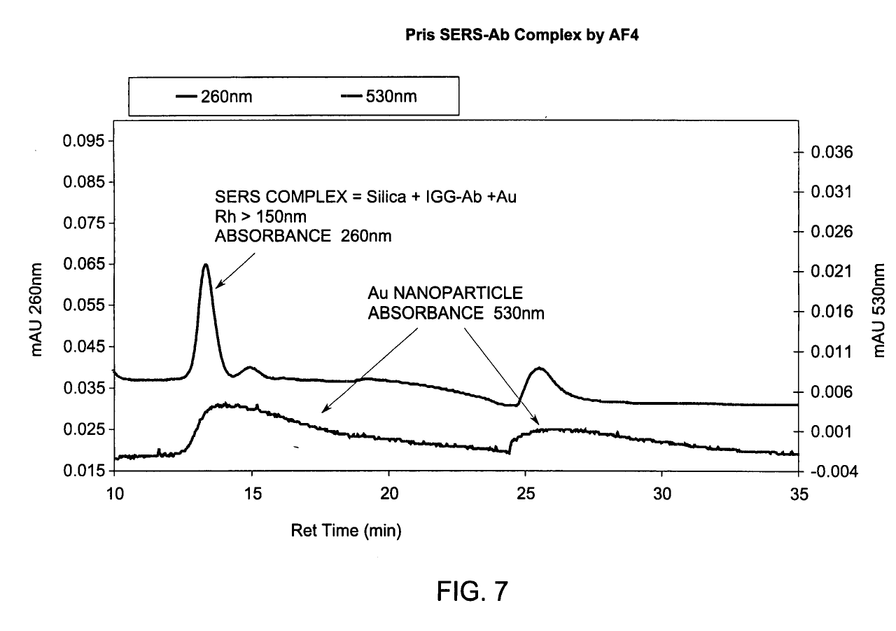

FIG. 7 depicts a field flow fractionation (FFF) chromatograph. This technique is capable of resolving/separating materials that have different cross-section in a single separation solvent. This isocratic (one solvent) separation allows for the avoidance or harsh organic chemicals, which are traditionally, used in gradient solvent separation schemes. The FFF separation scheme will have species with larger cross section elute first followed by species with smaller cross sections. The eluted materials are then characterized within this Example by two wavelengths of light: 260-nm and 530-nm. The selector absorbs light at 260-nm (upper line of chromatograph) while the analytical transducer nanoparticle used in this example will adsorb at 530-nm (lower line in chromatograph). Analysis of FIG. 7 leads to the conclusion that the larger complex elutes with both the selector and analytical transducer nanoparticle upon its surface (12 minute retention time), while the smaller individual components elute at greater than 25 minutes. Again, this illustrates that both the selector and the analytical transducer nanoparticle are coupled to the carrier particle surface as described in this invention.

### Example 5

### Use of the Transducer-Selector-Carrier in an Assay

The ability of the present method to create a complex that possesses stability and increased signal per selection event, while maintaining the analytical transducer nanoparticles and selector activity, allows for usage of the analytical transducer nanoparticles in various assay schemes. If the analytical transducer nanoparticle particles described in Example 1 were attempted to be coupled directly to the selector though various methods (including streptavidin/biotin, thiolated selector, non-specific cross-linking) the end product would include undesirable aggregates. This is especially true if the product particles were used in high stringency, high ionic strength buffers that are desirable to use in immunoassays. In this Example, the transducer-selector-carrier complex from Example 1 was used in conjunction with a standard immunomagnetic assay employing the desirable high stringency buffer without any of these issues.

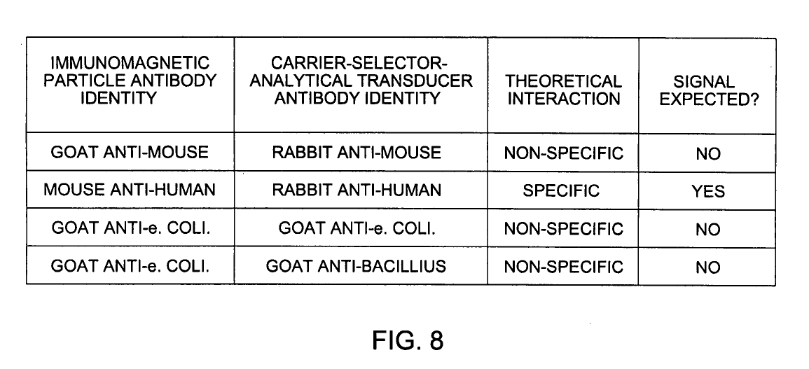

Activity of the selector along the analytical transducer nanoparticles is demonstrated using an immunomagnetic assay targeting paramagnetic beads. Streptavidin labeled MyOne™ beads (Dynal) were modified (by addition of biotinylated antibodies) according to vendor specifications. The MyOne™ beads were then assayed against various carrier particles-analytical transducer nanoparticles-antibody complexes in stringent, high ionic strength buffer (100 mM sodium phosphate, 150 mM sodium chloride, 1% BSA, 0.1% Tween-20 (pH 7.5)). The antibodies used as the selector for this immunoassay on each particle are shown in FIG. 8. Additionally, FIG. 8 also displays whether these selectors, and the particles that they are attached to, are expected to interact and provide a signal. More specifically, if the selector pairs were specific for each other, the MyOne™ beads would obtain a SERS signal from the analytical complex. The non-specific selector pair should be limited to no SERS signal.

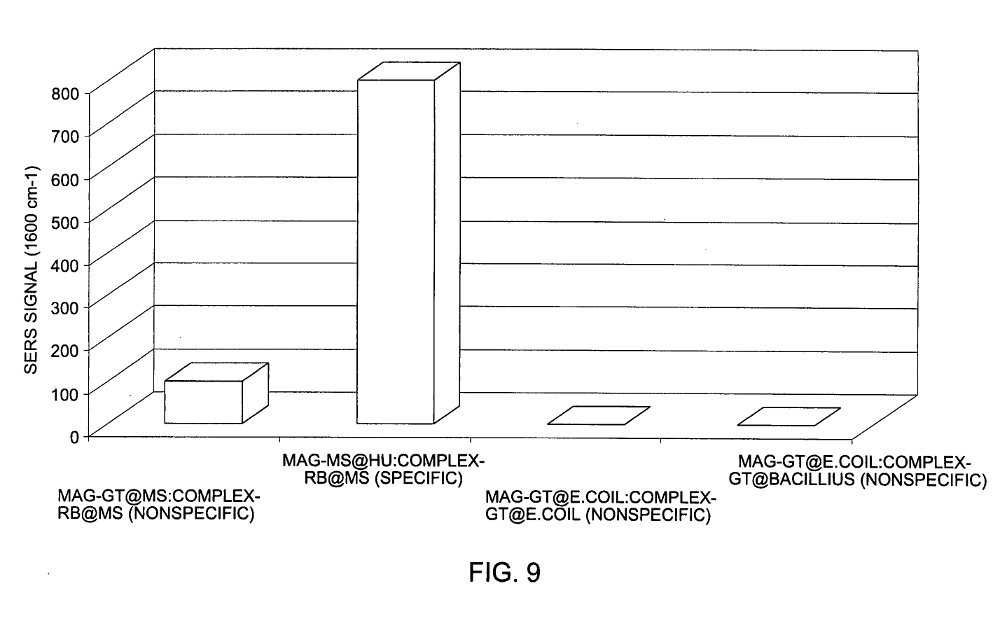

FIG. 9 displays the results of this assay and the empirical results are in line with the theoretical expectations. This is a result of a successful immunoassay allowed by the ability to use these complexes in high ionic strength buffers is advantageous because high ionic strengths prevent non-specific binding, thus advantageously reducing background effect in assays. The disclosed methodology of coupling analytical transducer nanoparticles to carrier particles and selector allows the resultant complex to be used in high ionic strength assay buffers, generating specific binding and sensitive assay conditions.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed as new and desired to be protected by Letters Patent of the United States is:

1. A composition, comprising:
a carrier;
an nanoparticle sized analytical transducer; and
a selector;
wherein said selector and said analytical transducer are linked to said carrier.

2. The composition of claim 1, wherein said carrier has a particle size at least as large as 100 nanometers.

3. The composition of claim 1, wherein said carrier is selected from the group consisting of alumina, iron oxide, titanium oxide, silica, glass, tin oxide, fluoropolymers, B-staged bisbenzocyclobutene monomers, phenolic resin, fluorinated poly(aryl ether sulfide), poly(isobutylene), poly(diphenoxyphosphazene), fluorinated acrylate, poly(methyl methacrylate), poly(vinyl alcohol), poly(vinyl butyral), poly(vinylcarbazole), poly(vinyl fluoride), poly(methyl vinyl ether), polyethylene, polypropylene, polystyrene, poly(vinyl pyridine), poly(ethylene oxide), fluorinated acrylates, poly(siloxanes), poly(silanes), poly(diphenoxyphosphazenes), poly(vinyl ferrocene), polycarbonates, poly(cyclic olefin), copolymers of the aforementioned polymers, blends comprising at least one of the aforementioned polymers.

4. The composition of claim 1, wherein said carrier is functionalized with an organic functional group.

5. The composition of claim 4, wherein said organic functional group is selected from the group consisting of acetals, ketals, acetylenic linkages, halides, alcohols, aldehydes, ethylenic linkages, esters, amides, amines, carboxylic acids, carboxylic anhydrides, azo groups, boranes, carbamates, epoxides, glycidyl ethers, glycidyl esters, thioethers, thiols, disulfides, cyano linkages, isothiocyanates, isocyanates, nitro groups, sulfonyl halides, sulfoxides, phenols, thiophenols, aromatics, hydrazides, aryl azides, nitrenes, imidoesters, benzophenones, carbonyldiimidazoles, carbodiimides, aziridines, alkylphosphates, siloxanes, and combinations of the foregoing.

6. The composition of claim 1, wherein said analytical transducer is sized less than 50 nanometers.

7. The composition of claim 1, wherein said analytical transducer comprises nanoparticles that exhibit transduction properties.

8. The composition of claim 1, wherein said analytical transducer is functionalized with an organic functional group.

9. The composition of claim 8, wherein said organic functional group is selected from the group consisting of acetals, ketals, acetylenic linkages, halides, alcohols, aldehydes, ethylenic linkages, esters, amides, amines, carboxylic acids, carboxylic anhydrides, azo groups, boranes, carbamates, epoxides, glycidyl ethers, glycidyl esters, thioethers, thiols, disulfides, cyano linkages, isothiocyanates, isocyanates, nitro groups, sulfonyl halides, sulfoxides, phenols, thiophenols, aromatics, hydrazides, aryl azides, nitrenes, imidoesters, benzophenones, carbonyldiimidazoles, carbodiimides, aziridines, alkylphosphates, siloxanes, and and combinations of the foregoing functionalities.

10. The composition of claim 1, wherein said selector is chosen from a group consisting of ligands, amino acids, antibodies, proteins, nucleic acids, enzymes, and combinations thereof.

11. The composition of claim 1, wherein the linkage between moieties of the composition has a disassociation constant smaller than 10⁻³M.

12. The composition of claim 11, wherein the linkage occurs through at least one of alkane, alkene, alkyne, amide, amine, hydazone, Schiff base, amidine, arylamine, carbamate, isourea, ester, disulfide, isothiourea, thioester, ether, epoxide, epoxy, thioether, sulfonamide, phosphoramidate, combinations of the foregoing bonds, hydrophobic interactions, electrostatic interactions and van der Waals interactions.

13. An analytical device comprising the composition of claim 1.

14. A method of making a composition, comprising:
providing a carrier, a analytical transducer sized in the nanoscale range and a selector; and
reacting the carrier with the analytical transducer and the selector to form a link between (i) the carrier and the selector and (ii) the carrier and the analytical transducer.

15. The method of claim 14, wherein the carrier has a particle size at least as large as 100 nanometers.

16. The method of claim 14, wherein the analytical transducer is sized less than 50 nanometers.

17. The method of claim 14, wherein said reacting the carrier and the analytical transducer give rise to a linkage that has a disassociation constant smaller than 10⁻³M.

18. The linkage of claim 17, wherein the linkage occurs through at least one of alkane, alkene, alkyne, amide, amine, hydazone, Schiff base, amidine, arylamine, carbamate, isourea, ester, disulfide, isothiourea, thioester, ether, epoxide, epoxy, thioether, sulfonamide, phosphoramidate, combinations of the foregoing bonds, hydrophobic interactions, electrostatic interactions and van der Waals interactions.

19. The method of claim 14, wherein said reacting the carrier and the selector give rise to a couple with a disassociation constant smaller than 10⁻³M.

20. The linkage of claim 19, wherein the linkage occurs through at least one of alkane, alkene, alkyne, amide, amine, hydazone, Schiff base, amidine, arylamine, carbamate, isourea, ester, disulfide, isothiourea, thioester, ether, epoxide, epoxy, thioether, sulfonamide, phosphoramidate, combinations of the foregoing bonds, hydrophobic interactions, electrostatic interactions and van der Waals interactions.

21. A device comprising a carrier, a nanoscale sized analytical transducer and a selector, wherein said selector and said analytical transducer are linked to said carrier.

22. The device of claim 21, wherein said carrier has a particle size greater than 100 nanometers.

23. The device of claim 22, wherein said carrier is selected from the group consisting of alumina, iron oxide, titanium oxide, silica, glass, tin oxide, fluoropolymers, B-staged bisbenzocyclobutene monomers, phenolic resin, fluorinated poly(aryl ether sulfide), poly(isobutylene), poly(diphenoxyphosphazene), fluorinated acrylate, poly(methyl methacrylate), poly(vinyl alcohol), poly(vinyl butyral), poly(vinylcarbazole), poly(vinyl fluoride), poly(methyl vinyl ether), polyethylene, polypropylene, polystyrene, poly(vinyl pyridine), poly(ethylene oxide), fluorinated acrylates, poly(siloxanes), poly(silanes), poly(diphenoxyphosphazenes), poly(vinyl ferrocene), polycarbonates, poly(cyclic olefin), copolymers of the aforementioned polymers, blends comprising at least one of the aforementioned polymers.

24. The device of claim 21, wherein said analytical transducer has a particle size less than 50 nanometers.

25. The device of claim 24, wherein said analytical transducer nanoparticle is selected from the group consisting of nanoparticles that possess transduction properties.

26. The device composition of claim 21, wherein said selector is chosen from the group consisting of ligands, amino acids, antibodies, proteins, nucleic acids, enzymes, and combinations thereof.

27. The device of claim 21, wherein said carrier and said analytical transducer are linked through at least one of alkane, alkene, alkyne, amide, amine, hydazone, Schiff base, amidine, arylamine, carbamate, isourea, ester, di-sulfide, isothiourea, thioester, ether, epoxide, epoxy, thioether, sulfonamide, phosphoramidate, and combinations of the foregoing.

28. The device of claim 21, wherein said carrier and said selector are linked through at least one of alkane, alkene, alkyne, amide, amine, hydazone, Schiff base, amidine, arylamine, carbamate, isourea, ester, di-sulfide, isothiourea, thioester, ether, epoxide, epoxy, thioether, sulfonamide, phosphoramidate, and combinations of the foregoing.
